(19) United States
(12) Patent Application Publication
(10) Pub. No.: US 2007/0027507 A1
(43) Pub. Date: Feb. 1, 2007

(54) Sensor configuration

(75) Inventors: Alison Burdett, Abingdon (GB); Paul Padden, Abingdon (GB)

Correspondence Address: ARENT FOX PLLC, 1050 CONNECTICUT AVENUE, N.W., SUITE 400, WASHINGTON, DC, 20036, US

(73) Assignee: Bio-Nano Sensium Technologies Limited

(21) Appl. No.: 11/403,157

(22) Filed: Apr. 13, 2006

(30) Foreign Application Priority Data
Apr. 26, 2005 (GB) 0508377.9

(51) Int. Cl. A61N 1/00

(52) U.S. Cl. 607/60

(57) ABSTRACT

Apparatus for monitoring and or treating a substance or body. The apparatus comprises a sensor 1, at least a portion of which 6 is arranged in use to come into contact with a body, tissue, bodily fluid or other substance, and a computer device 3. Both the sensor 1 and the computer device 3 have radio frequency circuitry 12,16 for facilitating the transmission of data between them via a wireless communication link, the computer device 3 having means 15,17 for selecting wireless link transmission properties based upon user location, and for signalling these properties to the sensor 1.

### FIELD OF THE INVENTION

The present invention relates to the configuration of sensors and in particular, though not necessarily, to the configuration of body implantable and wearable medical biosensors.

### BACKGROUND TO THE INVENTION

A “biosensor” has been defined as an analytical device incorporating a biological or biologically-derived sensing element either integrated within or intimately associated with a physicochemical transducer. Biosensors are generally designed to produce either discrete or continuous digital electronic signals that are proportional to a single analyte or a related group of analytes, although the provision of analogue signals should not be excluded.

There are many areas of application for biosensors including for example environmental sensing, chemical production, and food and drink production and preparation. One area of application that has attracted a great deal of interest however is that of medical diagnostics, monitoring, and treatment. The following discussion addresses primarily these medical applications, although it will be appreciated that the problems and solutions considered may also have non-medical applications.

A typical example of a medical monitoring biosensor is the glucose biosensor that is designed to produce an electrical signal indicative of the level of glucose present in a user's (i.e. the patient's) system. Today's glucose biosensors tend to be based around the concept of immobilising an enzyme or other reagent on the surface of an electrode to provide what is essentially a pH detector. When the reagent is exposed to a sample obtained from the patient, e.g. a drop of blood, the electrical output of the device indicates the pH value of the sample and hence indirectly the level of glucose. Commercially available glucose biosensors tend to be handheld type devices which accept a disposable test strip or element.

A user may be expected, e.g. in the case of a diabetes sufferer, to test his or her glucose level several times a day in order to provide a sufficient degree of feedback to allow intervention if the detected level deviates significantly from the “normal” level. Biosensors of this type have their limitations. In particular, due to the need for users to prick their skin to obtain a blood sample, and to then perform a short but still inconvenient test procedure using the biosensor, users may not perform the test as often as required. Skin pricking is also painful and, over the long term, can result in serious skin damage. These problems apply equally to other types of biosensors which measure analytes present in blood and thus require the provision of blood samples; for example the measurement of oxygen, lactate, nitric acid, creatinie, dopamine, serotonin, noradrenaline. The measurement of these analytes is useful in the understanding and monitoring of diseases as diverse as heart disease, rheumatoid arthritis and Parkinsons disease.

It is now appreciated that a solution to the problems considered in the previous paragraphs is to provide biosensors that are either implantable or wearable on a patient's skin, and many researchers are working towards producing such devices and systems. As well as giving feedback to users and clinicians, biosensors that are able to provide substantially continuous monitoring of a given condition offer the prospect of closed loop treatment systems, where treatment is applied in direct response to the monitored values. For example, proposals have been made and systems produced that inject insulin into a patient's system in response to the detection of a low blood sugar level. Both types of sensor, implantable and wearable, are likely to have their own distinct advantages, and will be used in different circumstances and to monitor and treat different conditions.

A number of factors are likely to be key to the successful development of commercially viable implantable and wearable biosensors. Chief amongst these is the need for low power consumption. Particularly in the case of implantable sensors, battery life must be extremely long, as surgical intervention would be required to replace a battery. In addition to minimising device power consumption levels, consideration has been given to powering devices using the electrochemical reaction of bodily substances, and even using electric and magnetic fields generated by the body (so-called energy scavenging techniques). In the case of wearable sensors that are likely to be disposable, low cost (e.g. a few pounds (British) or less) is also a priority. Where it is necessary to transmit data between the sensor and some monitoring and/or control system, consideration must be given to the (wireless) transmission mechanism, for example to minimise power consumption and to satisfy regulatory requirements.

U.S. Pat. No. 6,441,747 describes a wireless programmable system for medical monitoring that includes a base unit designed to communicate with a plurality of worn biosensor transceivers.

US2004/0096959 describes a glucose sensor in the form of a skin patch having a microneedle which penetrates the skin to draw out interstitial fluid. Glucose measurements are sent from the patch to a remote display unit, over a wireless link.

Other documents relevant to this field are:
IEEE Trans Biomed Eng, vol 35, no 7, July 1988, p 526-532;
Diabetes Technol Ther, vol 1, no 3, 1999, p 261-6;
Med Eng Phys, vol 18, no 8,1996 December, p 632-40;
US20010041831; and
WO2000067633.

### SUMMARY OF THE INVENTION

The present invention springs from a recognition that users of implantable or wearable biosensor utilising a wireless data transmission system to transfer data between the sensor and some local, e.g. belt worn or pocket held computer, will want to have the freedom to move across political borders and more particularly between regions and countries having different radio transmission regulations. As such, some means must be provided for allowing users to set the appropriate wireless link requirements, or for allowing these to be set essentially automatically.

It is also recognised that the desire to facilitate roaming applies equally to other medical sensor types, including chemical, electrical, and magnetic sensors, as well as to non-medical sensors, e.g. a pH sensor used to track meat over countries or between abattoirs.

According to a first aspect of the present invention there is provided apparatus for monitoring a property of a substance or body, the apparatus comprising:
a sensor, at least a portion of which is arranged in use to come into contact with said substance or body; and
a computer device,
   both the sensor and the computer device having radio frequency circuitry for facilitating the transmission of data between them via a wireless communication link, the computer device having means for selecting wireless link transmission properties based upon user location, and for signalling these properties to the sensor.

According to a second aspect of the present invention there is provided apparatus for monitoring and or treating a medical condition, the apparatus comprising:
a sensor, at least a portion of which is arranged in use to come into contact with a user's body, tissue, or bodily fluid; and
a computer device,
   both the sensor and the computer device having radio frequency circuitry for facilitating the transmission of data between them via a wireless communication link, the computer device having means for selecting wireless link transmission properties based upon user location, and for signalling these properties to the sensor.

Embodiments of the present invention allow the properties of the wireless link to be configured according to the location of the user. As the user roams between regions having different regulatory requirements, the properties of the wireless link can be changed or adjusted accordingly. This ensures that the equipment meets the local regulatory requirements and, more importantly, reduces the risk that environmental “noise” will interfere with the wireless link.

The properties selected by the computer system may comprise one or more of the following:

Available radio frequency spectrum;

Radio frequency carrier frequency;

Modulation scheme;

Channel bandwidth;

Transmission power;

Data rate;

Bias Voltage;

Bias Current;

Control signals;

Data signals.

The computer device may comprise a user interface allowing a user to select a location, e.g. from a list of options, the device having a processor for receiving the selection and for extracting the appropriate parameters from a memory of the device.

In an alternative embodiment, the computer device comprises means for detecting the location of the user. This could comprise any Global Navigation Satellite System (GNSS) receiver for example Global Positioning System (GPS), cellular telephone apparatus, or a radio receiver for receiving broadcast radio data. Alternatively, the means could comprise means for communicating with a discrete Global Positioning System receiver, cellular telephone apparatus, or a radio receiver held separately by the user.

The sensor may comprise one or more of the following:
A computer processor;
A computer memory (RAM or ROM or both);
A radio frequency receiver and or transmitter or both (a transceiver);
Signal processing circuitry;
Data conversion circuits;
Bias and control circuits;
Signal processing algorithms in hardware or software.

The computer device may comprise one or more of the following:
A computer processor;
A computer memory (RAM or ROM or both);
A radio frequency receiver and or transmitter or both (a transceiver);
A user interface;
Data conversion circuits;
Signal processing algorithms;
A display device;
An input device.

The computer device may comprise a cellular telephone, smartphone, personal digital assistant (PDA) or the like.

According to a third aspect of the present invention there is provided a method of monitoring and or treating a substance or body, the apparatus comprising:
using a sensor to monitor one or more parameters of the substance or body;
transmitting data between said sensor and a computer device via a wireless communication link; and
at the computer device, selecting wireless link transmission properties based upon user location, and signalling these properties to the sensor.

According to a fourth aspect of the present invention there is provided a method of monitoring and or treating a medical condition, the apparatus comprising:
using a sensor to monitor one or more parameters of a user's body, tissue, or bodily fluid;
transmitting data between said sensor and a computer device via a wireless communication link; and
at the computer device, selecting wireless link transmission properties based upon user location, and signalling these properties to the sensor.

The selected properties may be signalled to the sensor either directly, e.g. by sending parameter values to the sensor, or indirectly, by setting transmission properties of the computer device and allowing the sensor to detect the new in-use properties.

### BRIEF DESCRIPTION OF THE DRAWINGS

FIG. 1 illustrates schematically a monitoring system carried by a user;

FIG. 2 illustrates in cross-section a biosensor patch of the system of FIG. 1, attached to the user's skin;

FIG. 3 illustrates schematically, electronic components of the biosensor of FIG. 2;

FIG. 4 illustrates schematically a controller of the system of FIG. 1; and

FIG. 5 is a flow diagram illustrating a method of operating the system of FIG. 1.

### DETAILED DESCRIPTION OF CERTAIN EMBODIMENTS OF THE INVENTION

There is illustrated in FIG. 1 a human wearable monitoring system. This may be suitable, for example, for continuously monitoring the glucose level of a user suffering from diabetes. The system comprises two main components: a disposable sensor 1 in the form of a patch that is affixed to a user's skin, e.g. the arm 2, and a controller 3 which, in the example show, is attached to the user's belt 4. The controller 3 comprises a user interface including a liquid crystal display (LCD) 5 and a keypad 6.

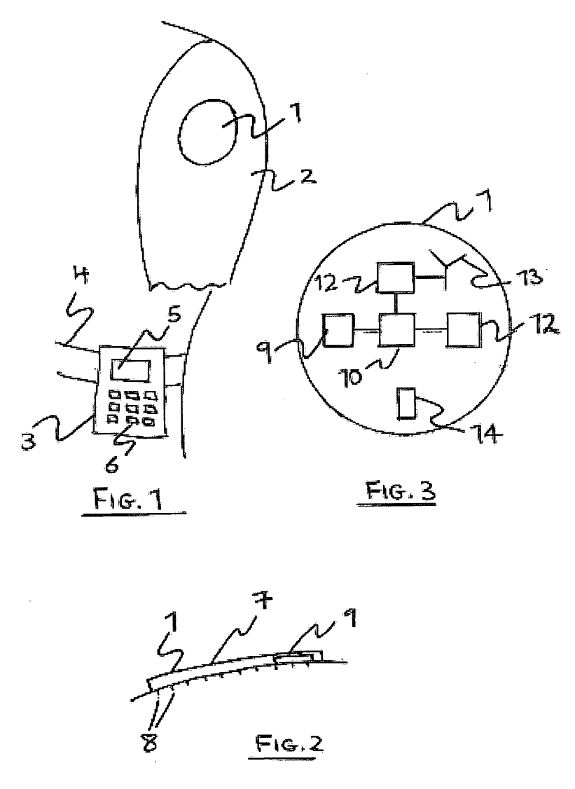

FIG. 2 shows a cross-sectional view of the sensor patch 1, affixed to the user's skin. The patch 1 comprises a flexible carrier 7 which may be of a plastics or fabric material, or of a metal foil. The underside of the carrier may be coated with an adhesive to allow the patch to be fixed to the skin, if the carrier is itself not sufficiently “sticky”. Projecting from the underside of the patch is an array, e.g. 100, of micro-needles 8. These are typically 1-1000 micrometers in diameter, and have the form of a hypodermic needle, i.e. with a passage extending through the middle thereof, the passage being open at the bottom tip. When the patch is pressed against the skin, the needles penetrate the surface of the skin down to the cutaneous level, allowing interstitial fluid to be conducted up through the needles into the patch. The relatively small size of the needles does not cause the user any pain, and apparently results in little or no long term skin damage. [See “ENDOPORATOR” (EU FP5 IST-2001-33141)].

Some means (not shown in the Figures) is provided for conducting fluid from the needles to an active biosensor component 9. This means could be, for example, a capillary tube or set of tubes, or a wick of some kind. The biosensor component 9 may be, for example, an ion sensitive field effect transistor (ISFET) based biosensor of the type described in “Weak Inversion ISFETs for ultra low power biochemical sensing and real time analysis”, Leila Shephard and Chris Toumazou, Sensors and Actuators 2004, Elsevier BV. Regardless of the type of biosensor used, the sensor will provide at an output an electrical signal that is indicative of the glucose level in the sampled fluid.

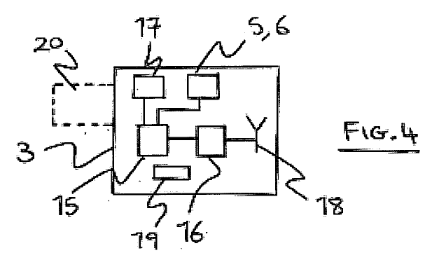

Referring now to FIG. 3, the various components of the sensor patch 1 are illustrated schematically. A processor 10 has an input coupled to the output of the biosensor 9. The processor 10 is also coupled to a memory 11 and to a radio frequency transceiver 12. The transceiver is coupled to a radio frequency antenna 13. Whilst the various components 9-13 may be provided as discrete components, in a preferred implementation these are all integrated onto a single piece of silicon. A power source 14 is provided to power the various electrical components. This could be, for example, a battery. For a new patch, the battery may be activated by the user tearing a strip from the patch, the strip isolating the battery terminals from the power supply leads.

Due to the need for small size and low cost, driven in turn by the requirement to provide a disposable patch, the complexity of the patch electronics must be kept to an absolute minimum. This objective also goes a long way towards satisfying 9 the requirement for extremely low power consumption. Very little processing is typically carried out on the raw monitored data by the patch electronics. The raw data may be merely digitised by the processor 10 and transmitted by the transceiver 12 over the wireless link, to the controller 3.

The main components of the controller 3 are illustrated schematically in FIG. 4. These include a microprocessor 15 coupled to a transceiver 16, a memory 17, and the user interface 5,6. The transceiver 16 is coupled to an antenna 18. These components are powered by a battery 19. It will be appreciated that the size and power consumption requirements placed on the controller are significantly less than those placed on the sensor 1. The approach used in this system is therefore to carry out most of the processing on the monitored signal at the controller 3. This will make use of some processing routines stored as program code in the memory 17 and accessed by the processor 15.

Different countries are likely to specify different requirements for the wireless link used to communicate between the controller 3 and the sensor 1. Whilst some countries may agree on a common standard, e.g. the countries of the European Union, there are likely to be a number of different “standards” around the world. The different standards may for example specify the radio frequency spectrum that can be used for the wireless link. They may also, for example, specify the transmission scheme that can be used, e.g. CDMA, UWB, FM, etc. It is critical therefore, in order to meet the regulatory requirements and to avoid potentially serious interference of data sent over the wireless link, that the monitoring system is able to be configured according to the prevailing requirements.

This requirement is achieved by storing in the memory 17 of the controller 1 the parameters required for each region. This could be in the form of a look-up table that is accessed by a country name. The data may be factory set, but is preferably updateable by some means. This could be via a USB interface to a pc having web access, or via a digital radio receiver tuned to some specific broadcast channel on which the required data is broadcast at regular intervals. Using the keypad 6 and LCD display 5, the user is able to enter a set-up mode 10 of the controller 3. The user selects his or her current location, and accepts the selection. The processor 15 then queries the look-up table stored in the memory 17, and obtains the appropriate configuration data. The transceiver 16 is configured appropriately. The processor 15 then causes the necessary configuration data to be sent over the wireless link to the sensor patch 1. The transceiver 12 of the sensor patch 1 is then configured accordingly, and can begin sending (monitored) data to the controller 3 for processing (and display).

Manual selection of the user's current location is relatively simple to implement, but has the disadvantage that a user is put at risk if he or she forgets to update the location, and continues to use a previous location, as well as contravening transmission regulations. A solution is to provide the controller with some means for automatically detecting a user's location. This could be achieved for example by integrating a GPS receiver into the controller 3. The GPS receiver provides to the processor 15 the current position, longitude and latitude and time, of the user. A GPS receiver 20 is illustrated in FIG. 4 using dashed lines. The memory 17 stores the information necessary to convert the position data into country identity, or directly into wireless link configuration parameters. As an alternative to a GPS receiver, a cellular telephone receiver may be provided, the receiver identifying the location based, for example, on the available cellular operators.

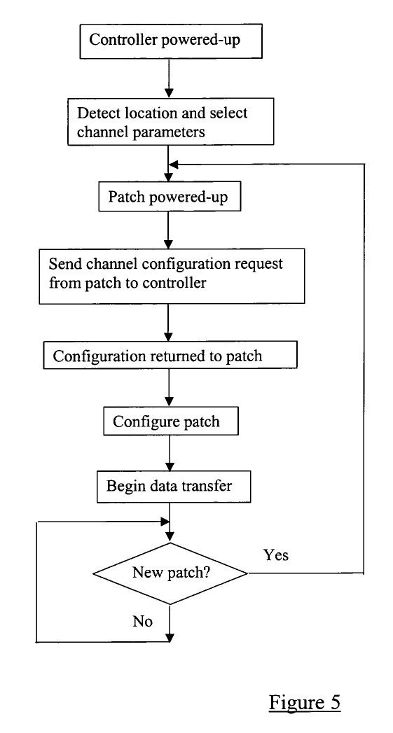

FIG. 5 is a flow diagram showing the general operating procedure of a monitoring system implementing the automatic location detection procedure (i.e. GPS-based). The user first powers-up the controller 3. As part of the self-initialisation process, the controller determines from the GPS module and the look-up table stored in the memory, the location of the user. This is stored in the memory. The location data may be refreshed periodically to ensure that any region change that occurs when the controller is on, are detected. The user powers-up the biosensor patch 1 by removing a foil tab which isolates the battery from the sensor electronics. As soon as power is applied to the electronics, the sensor controller 10 enters a configuration process. This causes the patch to enter into a receive mode, awaiting a configuration parameter command from the controller, sent using some default wireless communication channel. The controller transmits to the patch via the default channel, the parameters associated with the identified location. The patch configures its transceiver 12 accordingly, and future communications between the patch and the controller are conveyed by the “new” wireless channel. Instructions to change the channel may be sent by the controller to the patch, over this new link. Successful activation and configuration of the patch may be indicated by some form of indicator on the patch, e.g. LED, or by the sending of a feedback signal from the patch to the controller, the controller then providing an appropriate indication on its display. According to an alternative approach, following power-up of the controller, the controller may broadcast periodically on the wireless channel appropriate for the current location. Upon power-up of the sensor patch, the sensor scans a range of channels, modulation types, etc, to detect the broadcast. Once detected, the patch configures to this channel.

It will be appreciated by the person of skill in the art that various modifications may be made to the above described embodiment without departing from the scope of the present invention. For example, whilst the electronic and electrical components of the patch 1 and controller 3 have been illustrated as discrete functional entities, e.g. processor transceiver, memory, these entities may be merged together, at least to some extent. For example, certain of the functions of the transceiver may be implemented by the processor.

1. Apparatus for monitoring a property or condition of a substance or body, the apparatus comprising:
a sensor, at least a portion of which is arranged in use to come into contact with said substance or body; and
a computer device,
both the sensor and the computer device having radio frequency circuitry for facilitating the transmission of data between them via a wireless communication link, the computer device having means for selecting wireless link transmission properties based upon user location, and for signalling these properties to the sensor.

2. Apparatus for monitoring and or treating a medical condition, the apparatus comprising:
a sensor, at least a portion of which is arranged in use to come into contact with a user's body, tissue, or bodily fluid; and
a computer device,
both the sensor and the computer device having radio frequency circuitry for facilitating the transmission of data between them via a wireless communication link, the computer device having means for selecting wireless link transmission properties based upon user location, and for signalling these properties to the sensor.

3. Apparatus according to claim 1 or 2, wherein the properties selected by the computer system comprise one or more of the following:
radio frequency spectrum
Radio frequency carrier frequency;
Modulation scheme;
Transmission power;
Data rate;
Bias Voltage;
Bias Current;
Control signals;
Data signals;
Channel bandwidth.

4. Apparatus according to claim 1 or 2 and comprising a user interface allowing a user to select a location, the device having a processor for receiving the selection and for extracting the appropriate properties from a memory of the device.

5. Apparatus according to any one of claims 1 or 2 and comprising means for detecting the location of the user.

6. Apparatus according to claim 5, said means comprising a Global Navigation Satellite System receiver, cellular telephone apparatus, or a radio receiver for receiving broadcast radio data.

7. Apparatus according to claim 5, said means comprising means for communicating with a discrete Global Navigation Satellite System receiver, cellular telephone apparatus, or a radio receiver held separately by the user.

8. Apparatus according to claim 1 or 2, said computer device comprising a memory storing a look-up table mapping locations to wireless link properties.

9. Apparatus according to claim 1 or 2, said sensor comprising one or more of the following:
A computer processor;
A computer memory (RAM or ROM or both);
A radio frequency receiver and or transmitter or both (a transceiver);
Signal processing circuitry;
Data conversion circuits;
Bias and control circuits;
Signal processing algorithms.

10. Apparatus according to claim 1 or 2, said computer device comprising one or more of the following:
A computer processor;
A computer memory (RAM or ROM or both);
A radio frequency receiver and or transmitter or both (a transceiver);
A user interface;
Data conversion circuits;
Signal processing algorithms;
A display device;
An input device.

11. A method of monitoring and or treating a substance or body or condition thereof, the apparatus comprising:
using a sensor to monitor one or more parameters of the substance or body;
transmitting data between said sensor and a computer device via a wireless communication link; and
at the computer device, selecting wireless link transmission properties based upon user location, and signalling these properties to the sensor.

12. A method of monitoring and or treating a medical condition, the apparatus comprising:
using a sensor to monitor one or more parameters of a user's body, tissue, or bodily fluid;
transmitting data between said sensor and a computer device via a wireless communication link; and
at the computer device, selecting wireless link transmission properties based upon user location, and signalling these properties to the sensor.

13. A method according to claim 12, wherein the selected properties are signalled to the sensor either directly, by sending parameter values to the sensor, or indirectly, by setting transmission properties of the computer device and allowing the sensor to detect the new in-use properties.
